(19) United States
(12) Patent Application Publication
(10) Pub. No.: US 2016/0081563 A1
(43) Pub. Date: Mar. 24, 2016

(54) SYSTEMS AND METHODS TO ESTIMATE OR MEASURE HEMODYNAMIC OUTPUT AND/OR RELATED CARDIAC OUTPUT

(71) Applicant: PhysioWave, Inc., Santa Clara, CA (US)

(72) Inventors: Richard M. Wiard, Campbell, CA (US); Laurent B. Giovangrandi, Palo Alto, CA (US); Gregory T. Kovacs, Palo Alto, CA (US)

(21) Appl. No.: 14/494,493

(22) Filed: Sep. 23, 2014

(51) Int. Cl. A61B 5/0285; A61B 5/029; A61B 5/00; A61B 5/021

(52) U.S. Cl. CPC A61B 5/0285; A61B 5/02125; A61B 5/029; A61B 5/6824; A61B 5/6828; A61B 5/7278

(57) ABSTRACT

Certain aspects of the instant disclosure are sensing and/or providing estimates for blood pressure or cardiac output by using time-synchronous communication (or correlation) between two sensors. Specific embodiments concern an arrangement of devices including a time-synchronous circuit and a sensor. The sensor is configured to obtain, at or near a lower-body/extremity location of the user, time-related data indicative of speed or transit time of a propagating pressure wave while the wave travels in an artery and down a leg of the user. The time-synchronous circuit is configured to correlate information corresponding to or derived from the time-related data in a time synchronous manner with other cardiovascular information. The cardiovascular information corresponds to or is derived from hemodynamic output from the user by another sensor located at or near an upper-extremity location or lower-extremity of the user.

### BACKGROUND

An increasing number of approaches seem to be emerging in recognition of the importance of being able to conveniently measure multiple vital signs such as heart rate, blood pressure, cardiac output, body temperature, and activity, using either wrist watch, armband, hand-held-to-head, head-worn, or ear-worn sensors using electrocardiography, photoplethysmography, acoustic, or ear ballistocardiogram (BCG) signals or the motion cardiogram (MoCG), respectively, using sensors mounted in a single location in each instance (a hand-held device or earbuds, respectively). For exemplary products using one or more such sensors, reference may be made to products manufactured by Scanadu, HeadSense Medical Ltd., and Cambridge, Mass.-based Quanttus. Among these vital signs, non-invasive cardiac output and blood pressure are clinically relevant for cardiovascular management and exercise monitoring; however, these are generally regarded as challenging to measure with reasonable accuracy. For example, it can be challenging to accurately provide an estimate of the mean arterial pressure (MAP) by measuring the systolic and diastolic pressures and calculating the MAP as is conventionally done with existing devices. The MAP is the average arterial pressure (also referred to as the perfusion pressure). According to certain applications, Mean Arterial Pressure for relevant populations has been characterized in terms of the MAP of an individual as being normally between 70 mmHg and 110 mmHg, and it is believed that a MAP above 60 mmHg is necessary to deliver enough oxygen to sustain the organs; otherwise the tissues and organs would become ischemic.

### SUMMARY

Certain aspects of the instant disclosure are directed to addressing the above and other issues concerning blood pressure and cardiac output estimation by using time-synchronous communication (or correlation) between two sensors.

More specific aspects of the instant disclosure are directed to improving upon blood pressure and cardiac output estimation by using upper body sensors (e.g., upper-body extremity sensors internal to conventional head-worn or wrist-worn devices, in combination with a separate sensor worn at or below the knee), to more accurately estimate the relevant pulse transit times influencing blood pressure and cardiac output (CO). For example, by using such sensors to measure changes in total peripheral resistance (TPR), autonomic response changes in the circulatory system can now be taken into account.

In connection with a specific embodiment, an apparatus (in the form of a system or arrangement of devices) includes a time-synchronous circuit and a sensor. The sensor is configured and arranged to obtain, at or near a lower-body (or lower-extremity) location of the user, time-related data indicative of speed or transit time of a propagating pressure wave while the wave travels in an artery and down a leg of the user. The time-synchronous circuit is configured and arranged to correlate information corresponding to or derived from the time-related data in a time synchronous manner with upper-body or lower body cardiovascular information. The upper-body or lower-body cardiovascular information corresponds to or is derived from hemodynamic output from the user by another sensor located at or near an upper-extremity location or lower-extremity of the user.

In the case of the ear-worn sensor, two improved cases would be represented by: (i) the subclavian, axillary, brachial and radial arteries in the case of a hand-located pulse arrival sensor, or (ii) the descending aorta, femoral, popliteal and tibial arteries in the case of an ankle or foot-located pulse arrival sensor. In the case of a hand-held-to-head sensor which already incorporates pulse arrival at the hand, the longer path to the foot would further improve accuracies of pulse transit time and pulse wave velocity (PWV) measurement. Central (e.g., aortic) and leg (femoral) PWV contain more relevant physiologic indications of vascular parameters influencing the pressures experienced at the heart. Signals from the proximal pulse sensor(s) and distal pulse sensor(s) are recorded simultaneously to allow measurement of the pulse transit time (PTT). These signals, combined with appropriate estimates of the relevant arterial transit path, allow computation of the PWV.

Distal pulse arrival sensors may include, but are not limited to photoplethysmogram (PPG) sensors in a handheld device responding to finger PPG, wearable bracelets, or cuffs on one or both legs (e.g., ankle bracelet pressure cuff, PPG, impedance plethysmography (IPG), or toe clip PPG, etc.). Another embodiment could use either the hand-held-to-head or ear-worn sensors in conjunction (simultaneously) with a ballistocardiogram (BCG)/IPG/PPG scale, to directly measure the PTT. With an ear-worn device, the BCG signals could be synchronized together for redundancy, or to reduce the overall test measurement time by having redundant BCG signals measured. The characteristic of the signal from the distal pulse measurement is in the ability to measure characteristics of the incident and reflecting waves in the pulse waveform. These attributes include amplitudes and timings of minima, maxima, maximum change, area, etc. The attributes of the distal sensor are used to determine: (i) the arriving pulse timing, and (ii) an indication of the total peripheral resistance.

One embodiment incorporates an electrode into two surfaces of the device such that one electrode contacts one measurement site (such as the left chest) and the other electrode contacts the palm of one hand (such as the right hand, which might be holding the device). An electrocardiogram (ECG) signal can be recorded between these two electrodes. This signal can be used as a coronary pulse departure time marker relative to a more distal pulse arrival sensor, including a finger PPG sensor in the handheld device or an ankle, foot or toe-mounted pulse arrival sensor.

The above discussion/summary is not intended to describe each embodiment or every implementation of the present disclosure. The figures and detailed description that follow also exemplify various embodiments.

### DESCRIPTION OF THE FIGURES

Various example embodiments may be more completely understood in consideration of the following detailed description in connection with the accompanying drawings, in which:

FIG. 1A shows an embodiment of the present disclosure with a sensor worn at or below the knee (e.g., the ankle) including a three-axis accelerometer with at least one plethysmogram sensor (e.g., PPG, pressure transducer, pressure transducer array or pressure cuff) to detect the arterial pulse propagation of the ankle, and a circuit for communicatively coupling such upper-body measurements for use by the lower-body sensor.

FIG. 1B shows a schematic drawing of an all-in-one apparatus, according to the instant disclosure, to measure vital signs with the sensors placed upon the head, with exemplary time traces for the ECG, BCG and ear PPG, and with a circuit for communicatively coupling such upper-body measurements for use by the lower-body sensor.

FIG. 1C shows a schematic of the conduit arteries, also according to the instant disclosure, from the aorta to the head and example sensor placements useful for determining the pulse arrival or pulse transit times, depicting an exemplary time trace of the pulse arrival time measured at the arm using the ECG and finger PPG, and with a circuit for communicatively coupling such upper-body measurements for use by the lower-body sensor.

FIG. 2 is a set of timing diagrams showing aspects of the example embodiments discussed in connection with FIGS. 1A, 1B and 1C of the present disclosure.

FIG. 3 shows a normalized plethysmogram signal obtained below the knee, depicting the incident wave amplitude (A1) and reflected wave amplitude (A2).

FIG. 4 is a table that depicts cardiac output distributions of various organs during rest and exercise.

While various embodiments discussed herein are amenable to modifications and alternative forms, aspects thereof have been shown by way of example in the drawings and will be described in detail. It should be understood, however, that the intention is not to limit the disclosure to the particular embodiments described. On the contrary, the intention is to cover all modifications, equivalents and alternatives falling within the scope of the disclosure including aspects defined in the claims. In addition, the term “example” as used throughout this application is only by way of illustration, and not limitation.

### DETAILED DESCRIPTION

Aspects of the instant disclosure are directed to methods, devices and systems for improving over, and/or facilitating more accurate estimates and measurements concerning physiologic parameters such as blood pressure and CO by measuring pulse arrival times (PAT) and PTT. Certain embodiments are described below as examples involving use of a first sensor arranged to obtain a first set of user measurements and a second sensor, distal from the first sensor, for obtaining additional physiologic user data. The first and second sensors collectively process the user data to facilitate accurate estimates and measurements concerning these above-noted physiologic parameters.

A first specific embodiment concerns an apparatus (in the form of a system or arrangement of devices) that uses a time-synchronous circuit and a sensor worn at or below the knee of the user. The sensor is configured and arranged to obtain, at or near a lower-body (or lower-extremity) location of the user, time-related data. This data is indicative of speed or transit time of a propagating pressure wave while the wave travels in an artery and down a leg of the user. The time-synchronous circuit is configured and arranged to correlate information corresponding to or derived from the time-related data in a time synchronous manner with cardiovascular information. This information can correspond to the user's upper-body cardiovascular information or the upper-body or lower-body cardiovascular information corresponding to or derived from hemodynamic output from the user by another sensor located at or near an upper-extremity location or lower-extremity location of the user.

In another embodiment of the present disclosure, the upper-body or lower-body cardiovascular information indicates blood pressure and/or cardiac output estimation of the user. Additionally, the circuit is configured and arranged to provide more accurate estimation of the blood pressure and/or cardiac estimation of the user than is provided by the upper-body artery information.

In a further embodiment of the present disclosure the upper-body or lower body cardiovascular information indicates blood pressure and/or cardiac estimation measured at a location at or near the head of the user.

Another embodiment of the present disclosure includes an apparatus that measures a user's upper-body or lower-body cardiovascular information indicative of blood pressure and/or cardiac estimation at a location at or near the hand or wrist of the user.

Further embodiments of the present disclosure include an apparatus that measures a user's upper body or lower cardiovascular information indicative of blood pressure and/or cardiac estimation where the lower-body location is below a knee of the user, at the ankle of the user, at the foot of the user or at the bottom of the foot of the user.

Another embodiment of the present disclosure is an apparatus wherein the lower-body location is at the bottom of the foot of the user and one of the sensors is a scale that includes the sensor.

In another embodiment, practical aspects of the instant disclosure are directed to (“all-in-one”) vital monitoring devices in which a sensor is used to measure signals in the head region to estimate parameters such as blood pressure and CO by measuring PAT and PTT. In certain embodiments, the instant disclosure targets limitations involving these measurements/estimates including: (i) the relatively small CO distribution to the head region versus the rest of the body; (ii) the highly variable total peripheral resistance (TPR) in the extremities that influences local pressure; and (iii) the relatively short artery length of the head region that requires high fidelity recording to correctly identify timings. In another embodiment of the present disclosure, by using another sensor as described herein, blood pressure and cardiac estimation is significantly improved relative to such stand-alone hand-held or head-worn (all-in-one) devices. More specifically, one type of all-in-one vital monitoring device uses a sensor in contact with the user's head. The accuracy of the measurements/estimates obtained therefrom are complemented, for improved accuracy, by using a secondary plethysmograph sensor (or sensor device) worn at a peripheral artery location (e.g., at the ankle, foot, foot bottom, or other area below the knee). By using this secondary device in time synchronous communication with the first device (sensor in contact with the user's head), the arrangement provides indications of the pulse arrival or pulse transit times (via second sensor). Additionally, this sensor provides continuous indications of changes in total peripheral resistance measured at the peripheral artery location. A data-processing circuit (e.g., external CPU or microcomputer integral with the arrangement of the first and second sensor devices) can process and correlate the information collected from this secondary device in a time synchronous manner with the first device.

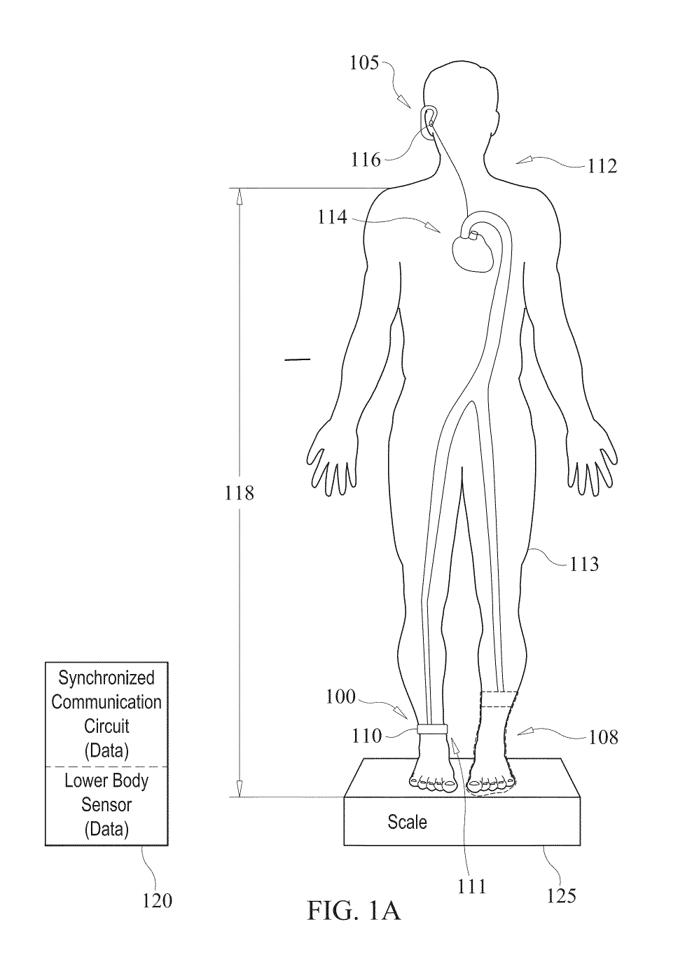

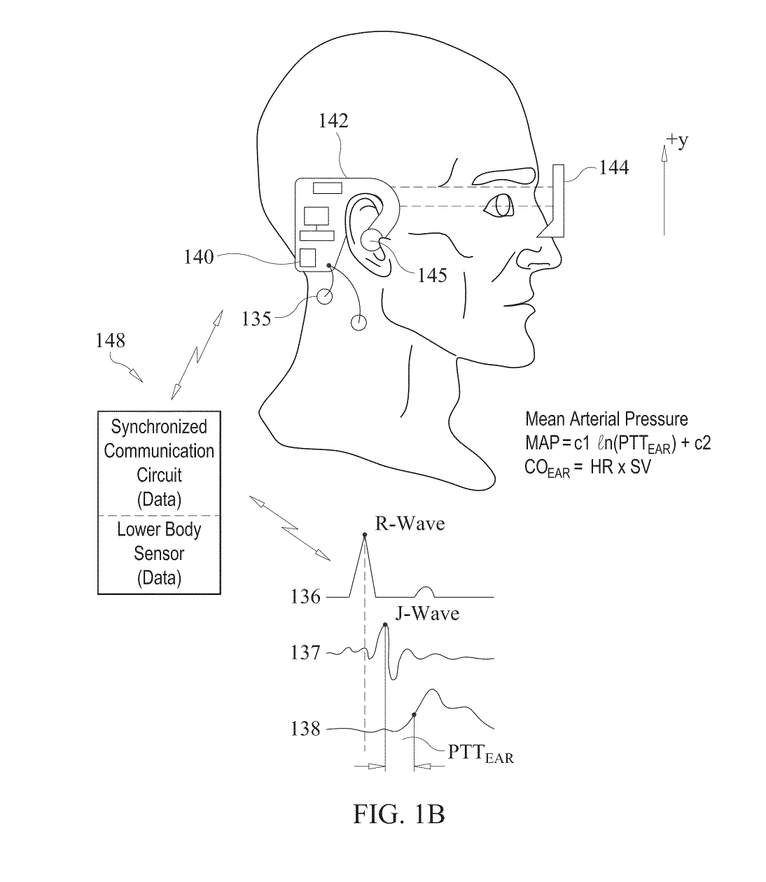

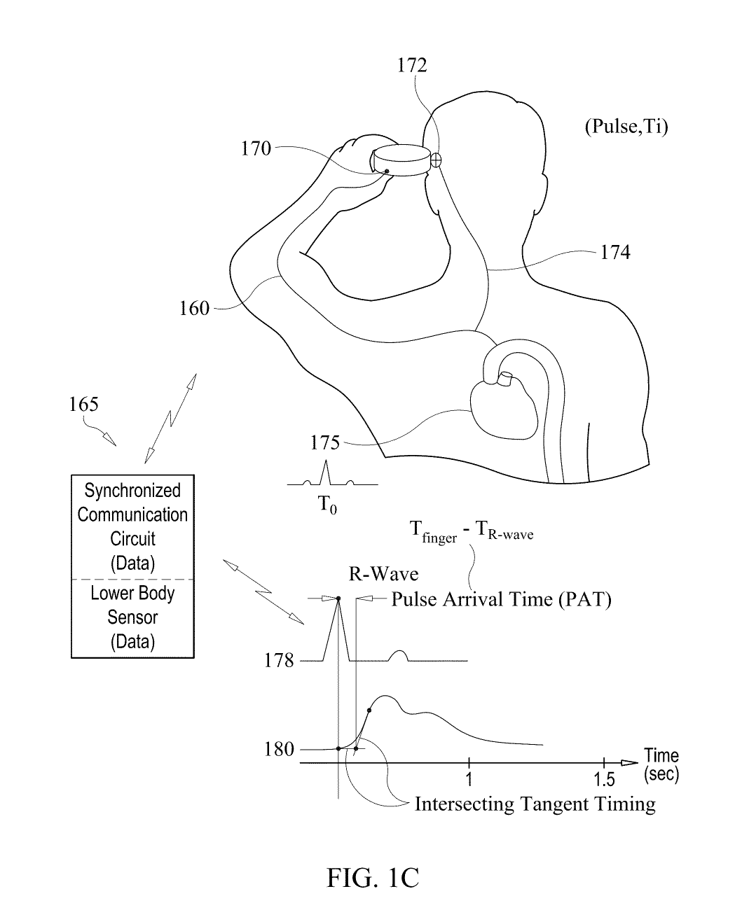

In an embodiment involving upper-body sensors (as shown in FIGS. 1A, 1B and 1C), speed, arrival times, and/or transit timings (e.g., time intervals) can be used to measure MAP. In one example, the PAT can be measured by determining the time interval between the ECG and a second pulse-derived signal (tonometric pulse, PPG, BCG, respectively), both measured at the head/neck region. The devices are directed towards making peripheral pressure PTT or pressure pulse time intervals. These intervals can be derived from the time intervals between the accelerometry BCG or MoCG, and the PPG or tonometric timings. The time delay (denoted as “PTT”) measured between a reference point between two points along an artery, such as a maxima, a minima, a point of maximum slope or the midpoint of the maxima and minima of the signal can be used. The PTT can be related to blood pressure (BP) via the equation based on the Moens-Korteweg and Hughes (or Bergel) blood pressure equations based on fluid dynamics and solid mechanics.

FIG. 1A shows an embodiment of the present disclosure directed at a system including a wearable device that can be worn on an extremity at or below the knee 113 (e.g., ankle) and which can also include a three-axis accelerometer 110 with at least one plethysmogram sensor 100 (e.g., PPG, pressure transducer, pressure transducer array, pressure cuff) to detect the arterial pulse propagation as sensed at the ankle FIG. 1A also illustrates embodiments including a distal extremity sensor 111 or an embodiment in which the lower-body sensor is in the form of a sock 108 worn by the user (the device shown on the right of the user's head is shown in more detail in FIG. 1B).

In further embodiments the lower body sensor includes a three-axis accelerometer 110 with at least one plethysmogram sensor (e.g., PPG, pressure transducer, pressure transducer array, pressure cuff) to detect arterial pulse propagation of the ankle or foot. FIG. 1A shows the distance between the user's heart and the user's lower extremities (e.g., ankle or foot) as the PTT distance 118. FIG. 1A also shows a synchronized circuit for communicatively coupling upper-body and lower body cardiovascular measurements for use by the lower-body sensor 120.

For certain embodiments consistent with FIG. 1A, the ankle band is comfortable and made with a conformal elastic or elastomer material to adjust its circumferential shape around the ankle due to the ambulatory motions of the user (e.g., walking, running) The material is breathable to regulate localized sweating that could interfere with sensor accuracy (e.g., fogging of the PPG optics). The device has a low-powered wireless transceiver that communicates to the all-in-one device 105 located at the head (head-located sensor).

As yet other alternatives (alone or in combination with one or more of the above-discussed sensors), the lower-body sensor can be at the bottom of the foot of the user, for example, in the form of a separate sensor (such as via electrode-equipped socks or foot-extremity engaging devices/attachments) or in a multi-purpose device such as foot sensor/platform such as a weighing scale shown. In FIG. 1A, such a scale 125 is depicted with a foot sensor/platform as the upper portion or surface region of the weighing scale in which the sensor electrodes reside for sensing at the bottom of the foot (the lower-body location). As an example weighing scale in which the sensor resides, reference may be made to foot electrodes (and related circuitry) as discussed in connection with U.S. Provisional Patent Application Ser. No. 62/011,466 (Impedance Measurement Devices, Systems, and Methods, Ref No. PHYW.005P1), which is incorporated herein by reference.

The ankle or foot-worn device wirelessly pairs with the head-worn device to first synchronize their internal clocks to determine the delay between the time dependent waveforms originating from the all-in-one head device and the propagating pulse arrival at the ankle sensor. An indicator light (e.g., LED) notifies the user that the pairing operation is complete. The plethysmography waveform of the ankle arterial pulse is measured by the sensor and is amplified and filtered by the analog circuitry. The resultant output is sampled by the analog to digital converter and stored on the internal memory with a timestamp calibrated to the clock of the head-worn device. The accelerometer 110 on the ankle worn-device detects motion data (e.g., walking, running, dancing) of the user and when motion is too excessive, and will stop recording plethysmogram signals until the user is quiescent to record signals with sufficient fidelity. In another embodiment, the signals will continue to be recorded while the user is ambulatory and the accelerometer motion signals are used as an error signal input as an adaptive filtering algorithm to process the plethysmogram waveform. The wireless ankle device transmits the recorded plethysmography waveform to the all-in-one device for signal processing to determine the PTT or PAT.

The transmitted time-stamped ankle-plethysmogram waveform is stored in the memory of the all-in-one head worn device for additional signal processing. The all-in-one device provides at least one signal to indicate the beginning of the cardiac cycle or proximal pressure pulse, using the ECG 112, BCG (MoCG) 114 or head (or ear) PPG 116 signal to determine the PTT or PAT. For example, the R-wave timings of the ECG are detected with a threshold peak-detection algorithm to indicate the start of the cardiac cycle. The intersecting tangent method is applied to the ankle plethysmogram to indicate the arrival time of the distal pulse. The timing interval is computed to determine the PAT. In another embodiment, the BCG or MoCG signals measured at the head are used to indicate the start of the proximal pressure pulse. A series of BCG or MoCG signals are collected and parsed into ensemble beats using the R-wave timing of the ECG as a timing reference for each beat. The BCG or MoCG beats are then ensemble averaged together to improved estimation of the BCG or MoCG which is commonly corrupted by motion artifacts when the user is moving. The I-wave or J-wave timing of the ensemble-averaged BCG (MoCG) signal is identified with a peak detection algorithm which represents the proximal pressure timing of the aortic arch. The ankle plethysmograph signal is ensemble averaged in a similar manner using the R-wave timings of the ECG. The intersecting tangent method is applied to the ankle plethysmogram signal to indicate the arrival time of the distal pulse. The PTT is then determined by calculating the timing interval between the proximal and distal pulse timings.

Some known equations that can be used for estimating blood pressure obtained from the PTT are depicted below. The example relates BP to PPT, using the Young's modulus (material parameter) of the artery wall. In other examples, the PAT is used instead of the PTT; however, PAT is generally known to be a less accurate measure since cardiac timings in the ECG vary significantly before the aortic valve opens to pressurize the aorta.

The PTT in an artery can be expressed using the Moens-Korteweg relationship for the PWV of a thin-walled artery of length (L), which are related by the material properties and geometry of the artery filled with blood, an incompressible fluid, as:

$$PWV = \frac{L}{PTT} = \sqrt{\frac{Eh}{2 ρ r}}$$

where the PWV is related to the Young's modulus (E) of the pressurized arterial wall, the wall thickness (h), the blood density (p), and the vessel radius (r). The Young's modulus (E) for an artery can be treated as linear or non-linear elastic depending on either small deformations (e.g., linear) or larger deformations (non-linear). Non-linear assumptions are reasonable for compliant arteries that undergo significant pressure changes between resting and exercise conditions. Therefore, the Young's modulus for a non-linear elastic material is:

E=E₀e^{αP}

Where E₀ and α can be characterized in stress-strain experiments. Since PWV is equal to the artery length (L) divided by the PTT, blood pressure can be solved for (estimated) as done previously by Bergel and Hughes:

P=c1 ln(PTT)+c2

where c1 and c2 are:

$$c 1 = \frac{- 2}{α}$$

$$c 2 = \frac{1}{α} ln ( \frac{2 {L}^{2} ρ r}{{E}_{0} h} )$$

where P is the MAP. Constants c1 and c2 can be solved for with blood pressure cuff calibrations. The equation above supports one example of blood pressure estimation derived from the PTT.

The Hughes equations derived for pressure and PTT are well correlated (e.g., r²>0.9) when measured under controlled lab conditions; however this BP/PTT correlation is not accurate when autonomic reflexes are considered. Clinical data has shown that the lack of correlation when subjects are successively tilted between supine and standing positions can be a limitation of current integrated vital sign monitors in the head region. As such, changes in pressure due to autonomic response changes in the peripheral vasculature require continuous estimations of the peripheral resistance, which is a key aspect of the present disclosure.

The present disclosure measures TPR continuously using a distal artery plethysmography sensor, which is in communication with an integrated sensor as described in the known equation to improve the accuracy of MAP and CO. MAP is also related to the cardiac output and TPR as:

MAP=CO×TPR=HR×SV×TPR.

where HR is the heart rate, SV is the stroke volume, and TPR is the total peripheral resistance, which is highly variable, due to vasodilation/vasoconstriction in arteriole bed of the extremities.

Therefore, CO can be expressed in terms of the PTT as:

$$CO = \frac{c 1 ln ( PTT ) + c 2}{TPR} .$$

The artery of interest in the known examples is located between the aortic arch and the head; such as the carotid artery, ear lobe, or superficial temporal artery, which is part of the cerebral blood flow tract, which all have limited correlative value to estimate MAP, since: (i) the artery along the neck is relatively short requiring high fidelity measurements, and (ii) the pressure to the brain is tightly regulated (e.g., constant PTT's), while pressures can vary significantly in the aortic and femoral arteries between rest and exercise (e.g., highly variable PTT's). Therefore, the MAP estimation would be improved over the known examples by measuring PPT along the aortic-femoral artery instead of the arteries of the head.

The derived PTT and PAT timing (intervals) used in the known examples are limited (e.g., limited accuracy and limited calibration time held) when attempting to determine blood pressure. First, the measured PTT is between the heart-to-head (ear, or temple). The blood flow to the head only carries 15-18% of the CO during rest and can decrease to 5% during exercise. In contrast, the majority of CO change is in the arteries delivering blood to the skeletal muscles and this change can be 15% of the CO during rest up to 65% of the CO during exercise. The major organs shunt blood to the muscles during periods of strenuous physical demand. Thus, the aorta and femoral arteries become the more relevant track to monitor blood pressure, cardiac output, and therefore PTT/PAT. Head-contact measurement devices would be improved in accuracy significantly by incorporating a secondary sensor to measure PTT that traverses the central arteries (aortic and femoral) to maximize sensitivity to detecting CO and blood pressure changes, in communication with said first sensor.

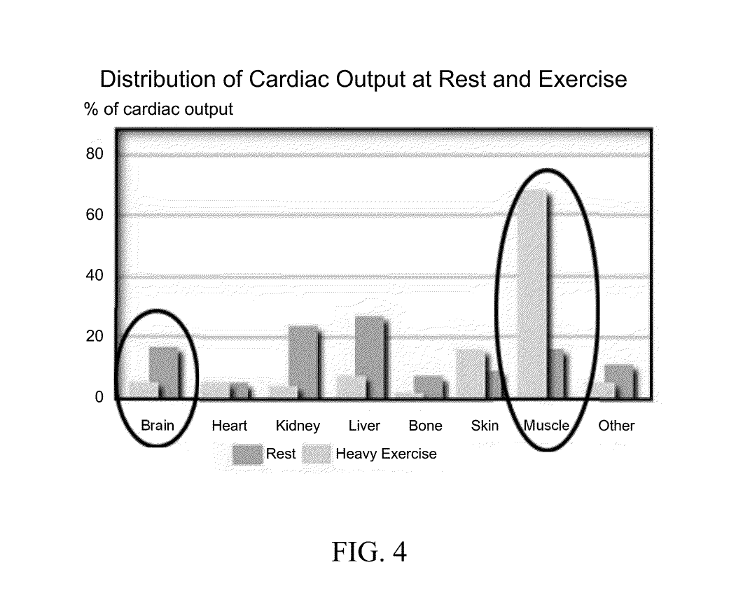

FIG. 4 is a table that depicts cardiac output distributions via various organs during rest and exercise as is known in connection with all-in-one head-worn devices (see, e.g.,

Montana State University-Bozeman website, Physiology and Psychology—Performance Benchmarks—Cardiac Output, at: btc.montana.edu/olympics/physiology/pb01 with the “Heavy Exercise” bars to the left of the “Rest” bars along the horizontal axis.

FIG. 1B shows a schematic drawing of an integrated (all-in-one) device to measure vital signs with the sensors placed upon the head featuring a wireless transceiver 142 that continuously determines vital signs (e.g., heart rate, heart rate variability, blood pressure, body temperature, SpO2, stroke volume, and activity) for a stationary or ambulatory person. The head-contact device is discreet, fashionable, compact, lightweight, and comfortably worn on the head. A general consumer can affix the monitor to the body as a worn accessory 144 (e.g., sunglasses, prescription glasses, audio headphones, and cell phone ear bud). The monitor can be used with or without an integrated video display to display vital signs in a static or continuous manner. The device can be worn comfortably and used while sitting down, standing still, or sleeping. The device can also be used for continuous monitoring of vital signs while exercising where heart rate, cardiac output, and activity is increased and decreased. FIG. 1B also shows a synchronized circuit for communicatively coupling upper-body and lower body cardiovascular measurements for use by the lower-body sensor 148. The bottom portion of FIG. 1B shows time aligning signal graphs for electrocardiogram (ECG) 136, BCG (MoCG) 137, and PPG (from the ear) 138 which collect data from the user. The ECG graph shows a high peak R-wave which indicates the beginning of the cardiac cycle. The BCG graph shows a high peak J-wave, which represents hemodynamic movements from the user's heart to the head and can help indicate what position the user is in. The PPG graph shows interaction between the signals and a time-delay indicative of aortic femoral PTT. The right side of FIG. 1B shows the MAP represented as:

MAP=c1 ln(PTTₑₐᵣ)+c2

The right side of FIG. 1B also shows the cardiac output as related to the user's ear as:

CO(Ear)=HR×SV

The known example uses the accelerometer and/or a gyroscope sensor 140 inside the device to suspend monitoring of vital signs to conserve power when activity is too excessive which reduces the signal integrity (or signal to noise) of the vitals. The device includes algorithms with pre-set and user-set alarms to provide an indication of achieving target ranges for vital signs, or to indicate a condition when vital signs are too high or too low. The device pairs to wireless peripherals (e.g., laptop, computer, smartphone or tablet) through a peer-to-peer or through a networked router connection to store, compute, and display results and trends to the user. The user can interact with the data and results using a website or dedicated app on the peripheral device using a graphical user interface (GUI) or speech input. The data from the device can also be stored on a memory card, or can be downloaded from the internal memory of the device using a serial data connection (e.g., USB) to a peripheral device.

In one embodiment of the present disclosure the user can input calibration information which is determined with a blood pressure cuff measurement into the device to store calibration constants (e.g., c1 and c2). These constants can be used to estimate blood pressure when used in conjunction with PTT or PAT measurements. In similar fashion, calibration information for CO can be stored to estimate TPR. The user may also input addition parameters such as age, gender, height, and weight, to further calibrate the blood pressure estimate. These additional calibration parameters can be retrieved from a look-up table, or computed using mathematical models (e.g., regression, transfer function, neural network, etc.).

The integrated device includes several sensors which are used to collect physiologic data from the user such as ECG (135), three-axis accelerometry, BCG (140), MoCG, respiration rate (145), SpO2, temperature, and PPG. The sensors sample the physiologic data using analog and digital circuitry to amplify and filter the raw signal into processed time-dependent waveforms using a microcontroller or co-processor. Timings, amplitudes, derivatives, and frequency content are computed using algorithms (e.g., peak-detect algorithms, fast Fourier transform (FFT), ensemble averaging, Finite Impulse Response (FIR)/Infinite Impulse Response (IRR) digital filters, to extract relevant features from the processed waveforms. The processed waveforms and computed features are stored on internal memory.

FIG. 1B and FIG. 1C respectively depict sensor arrangements on the head as used to measure the PAT and PTT. With each heartbeat the sensors are used to measure signals that are processed by the device. For example, each heartbeat generates electrical potentials that are detected with gel or dry electrode near the neck to generate a time-dependent ECG waveform. The ECG circuit is measured with analog and digital circuitry to capture a well-defined R-wave which can be used as the initial reference timing for the start of the cardiac cycle. Heart rate detection and heart rate variability can be determined from the ECG waveform using well-known algorithms and are stored in the memory of the device.

The head-worn BCG or MoCG sensor in FIG. 1B records the waveform produced by the accelerations of blood moving from the aortic arch up the carotid artery into the head and the timings and morphology of this acceleration waveform are subject-dependent. In similar fashion, the three-axis accelerometry signals are measured by an analog and digital circuit to amplify and reject common noise sources (e.g., 60 Hz line noise). The time-dependent BCG waveforms are further processed to determine the posture of the user. The y-axis (headward-footward) channels from the accelerometers are used to estimate the BCG time-dependent waveform. Further processing is done to determine if the subject was still enough during the recording to use the signals with minimal body-induced noise.

FIG. 1C depicts the user's temporal artery 172 and the user's carotid artery 174. This figure depicts the user's PAT in the arm 160 as the difference between the finger sensor and R-wave timing. An ECG signal 175 can also be used to indicate estimated CO. The bottom of FIG. 1C depicts a signal time aligning graph, showing ECG 178 and PPG 180 signals. The ECG graph shows a peak of R and a measurement of the PAT. The PPG graph shows an intersecting tangent that reflects the PAT value over time. FIG. 1C also shows a synchronized circuit for communicatively coupling upper-body and lower body cardiovascular measurements for use by the lower-body sensor 165.

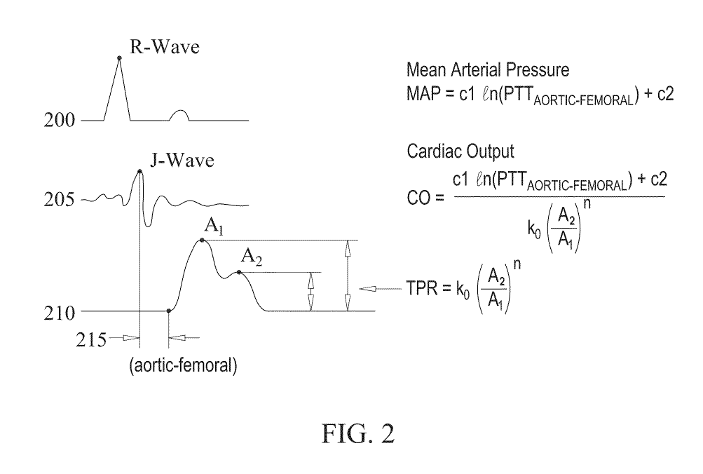

FIG. 2 shows three time aligning signal graphs, an ECG 200, BCG 205, and an ankle plethysmograph 210. These signals indicate the beginning of the cardiac cycle or proximal pressure pulse of a user. An ECG wave form is shown with a peak of R. This wave form is obtained by using a sensor on the user's upper body, (e.g., arm) as discussed in FIG. 1A. This wave form is also discussed in FIG. 1B. A BCG wave form is shown with a peak of J. The J-wave peak represents the proximal pressure timing of the aortic arch. This wave form is obtained by using a sensor at the user's ear, as discussed in FIGS. 1A and 1B. An ankle plethysmograph is also shown, indicating an intersecting tangent method, used to indicate the arrival time of the distal pulse and the total peripheral distance reflecting the difference between PA1 and PA2 as shown in FIG. 2 and relating to:

$$TPR = {{k}_{0} ( \frac{{A}_{2}}{{A}_{1}} )}^{n} .$$

The interaction of these signals measure PTT 215, showing aortic femoral time delay, to calculate cardiac output of a user.

For certain embodiments in which a lower-body sensor is used with previously-known upper-body sensors (as discussed herein), aspects of the present disclosure improve the accuracy of CO estimate and blood pressure determination, using timing and amplitude information obtained from the distal plethysmograph. The ensemble averaged ankle/foot plethysmograph is used to provide more sensitive PTT indications by directly measuring the arterial segment (aortic-femoral) that influences aortic blood pressure. Aortic blood pressure estimation using the Hughes equation is improved by providing a more relevant PTT measurement that influences aortic blood pressure since the PTT of the central and femoral arteries carry the primary reflecting wave back to the heart which is commonly known as the augmented pressure. The femoral arteries increase in caliber during exercise to direct more blood flow to the legs and the pressure will increase to deliver blood more quickly to meet metabolic demands in the leg muscles which are accompanied by an increase in heart rate (HR). The aortic-femoral PTT change is far more significant and correlated than PTT changes in the artery traveling to the head.

Furthermore, the present disclosure also provides indications of the TPR changes of this arterial segment which significantly impacts the accuracy of cardiac output estimates (CO). Since MAP is equal to:

MAP=c1 ln(PTT_{aortic-femoral})+c2

and MAP may also be expressed in terms of cardiac output

MAP=CO×TPR=HR×SV×TPR

therefore,

$$CO = \frac{c 1 ln ( {PTT}_{aortic \text{-} femoral} ) + c 2}{TPR}$$

where this expression for CO incorporates TPR. This form of CO determination differs significantly from the form taught by He, where the BCG or MoCG J-Wave amplitude was used to estimate stroke volume (SV) changes [regression: SV=20.4×(J-amplitude, mG), expressed in mL], which is obtained from the proximal sensor. In the present disclosure, CO is determined by measuring an indication of TPR measured by the distal sensor. TPR may be expressed by the resistance (R) in a long thin-walled artery as:

$$R = \frac{8 μ L}{{r}^{4}}$$

where (g) is the blood viscosity, (L) is the artery length, and (r) is the radius of the artery.

The time dependent ankle plethysmograph contains indications of TPR by examining the amplitude (A) differences between the incident (A1) wave normalized to unity and the reflecting wave (A2), which are measured at the ankle, wrist, or fingertip—wherever a waveform with reflection data is available. For example, at rest the amplitude ratio of the reflecting wave to the incident wave (A2/A1) may be 35% amplitude at rest and will decrease to 24% during exercise when the arterioles in the foot dilate. Therefore, an empirical form of TPR may be derived from the fluidic resistance expression (R) as:

$${TPR}_{empirical} = {{k}_{0} ( \frac{{A}_{2}}{{A}_{1}} )}^{n}$$

where k₀ and n are constants derived from a population dataset between resting and exercise. Therefore, CO in terms of a proximal and distal sensor arrangement may be expressed as:

$$CO = \frac{c 1 ln ( {PTT}_{aortic \text{-} femoral} ) + c 2}{{{k}_{0} ( \frac{{A}_{2}}{{A}_{1}} )}^{n}} .$$

The aortic-to-ankle PWV of the user can also be determined by taking the measured length between the proximal and distal sensors and dividing the length by the PTT. The length measurement can be measure directly by a tape measure or ruler and placed as a parameter (e.g., measurement constant) into the memory of the all-in-one device where the input is recorded and stored by one of the peripheral devices described previously. In another embodiment, the length between the proximal and distal sensors is estimated by the user's height, which uses a look-up table of proximal-distal distances that have been previously determined through statistical approaches applied to a reference population of users based on gender. The calculated body-length PTT (e.g., aorta to ankle, or aorta to foot) is then used as input to determine MAP as described previously. Blood pressure cuff measurements are used as a calibration parameter to determine constants c1 and c2.

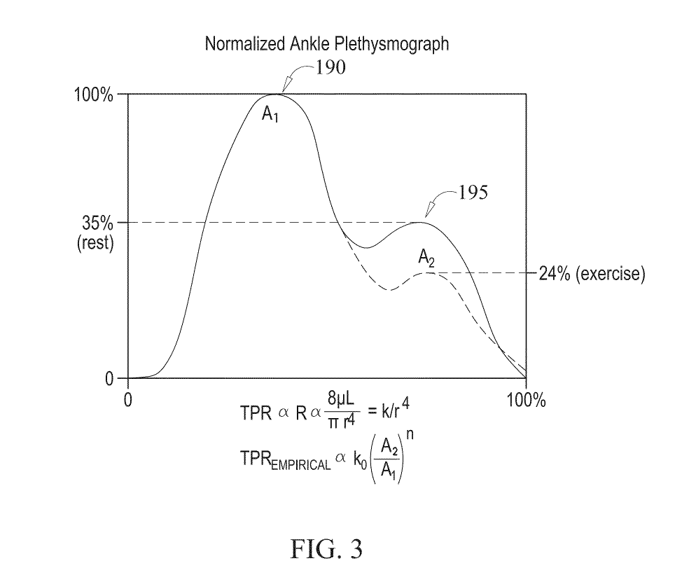

FIG. 3 shows a normalized ankle plethysmograph signal. This figured depicts the incident wave amplitude (A1) 190 and reflected wave amplitude (A2) 195. This figure depicts the TPR expressed by the resistance (R) in a long thin-walled artery as shown by:

$$TPR ∝ R ∝ \frac{8 μ L}{Π {r}^{4}} = K / {r}^{4} .$$

and the empirical total peripheral resistance as represented by the following equation:

$${TPR}_{empirical} ∝ {{k}_{0} ( \frac{{A}_{2}}{{A}_{1}} )}^{n} .$$

Various blocks, modules or other circuits may be implemented to carry out one or more of the operations and activities described herein and/or shown in the figures. In these contexts, a “block” (also sometimes “circuitry,” “logic” or “module”) is a circuit that carries out one or more of these or related operations/activities (e.g., using some form of sensing (e.g., photo or electrodes) to sense hemodynamic output, PWV, etc.). For example, in certain of the above-discussed embodiments, one or more modules are discrete logic circuits or programmable logic circuits configured and arranged for implementing these operations/activities, as in the circuit modules shown in FIGS. 1A, 1B and 1C. In certain embodiments, such a programmable circuit is one or more computer circuits programmed to execute a set (or sets) of instructions (and/or configuration data). The instructions (and/or configuration data) can be in the form of firmware or software stored in and accessible from a memory (circuit). As an example, first and second modules include a combination of a CPU hardware-based circuit and a set of instructions in the form of firmware, where the first module includes a first central processing unit (CPU) hardware circuit with one set of instructions and the second module includes a second CPU hardware circuit with another set of instructions. Further, certain embodiments are directed to a computer program product (e.g., nonvolatile memory device), which includes a machine or computer-readable medium having stored thereon instructions which may be executed by a computer (or other electronic device) to perform these operations/activities.

Based upon the above discussion and illustrations herein, those skilled in the art will readily recognize that various modifications and changes may be made to the various embodiments without strictly following the exemplary embodiments and applications illustrated and described herein. For example, it would be appreciated that various embodiments and applications would be advantaged by using multiple sensors (as upper-body and/or lower-body location-based sensors). In addition, the various embodiments described herein may be combined in certain embodiments, and various aspects of individual embodiments may be implemented as separate embodiments. Such modifications do not depart from the true spirit and scope of various aspects of the invention, including aspects set forth in the claims.

What is claimed is:

1. An apparatus comprising:
a sensor configured and arranged to obtain, at or near a lower-body (or lower-extremity) location of the user, time-related data indicative of speed or transit time of a propagating pressure wave while the wave travels in an artery and down a leg of the user; and
a circuit configured and arranged to correlate information corresponding to or derived from the time-related data in a time synchronous manner with upper-body or lower body cardiovascular information, the upper-body or lower-body cardiovascular information corresponding to or derived from hemodynamic output from the user by another sensor located at or near an upper-extremity location or lower-extremity of the user.

2. The apparatus of claim 1, wherein the upper-body or lower-body cardiovascular information is indicative of blood pressure and/or cardiac output estimation and the circuit is further configured and arranged to provide an estimation of the blood pressure and/or cardiac estimation to a higher degree of accuracy than is indicated by the upper-body artery information.

3. The apparatus of claim 1, wherein the upper-body or lower body cardiovascular information is indicative of blood pressure and/or cardiac estimation as measured at a location at or near the head of the user.

4. The apparatus of claim 1, wherein the upper-body or lower-body cardiovascular information is indicative of blood pressure and/or cardiac estimation as measured at a location at or near the hand or wrist of the user.

5. The apparatus of claim 1, wherein the lower-body location is below a knee of the user.

6. The apparatus of claim 1, wherein the lower-body location is at the ankle of the user.

7. The apparatus of claim 1, wherein the lower-body location is at the foot of the user.

8. The apparatus of claim 1, wherein the lower-body location is at the bottom of the foot of the user.

9. The apparatus of claim 1, wherein the lower-body location is at the bottom of the foot of the user and the sensor is a scale including the sensor.

10. An apparatus comprising:
a sensor configured and arranged to obtain, at or near a lower-extremity location of the user, time-related data indicative of speed or transit time of a propagating pressure wave while the wave travels in a peripheral artery down a leg of the user; and
a circuit configured and arranged to:
correlate information corresponding to or derived from the time-related data in a time synchronous manner with upper-body or lower body cardiovascular information, the upper-body or lower body cardiovascular information corresponding to or derived from physiologic cardiac output from the user by another sensor located at or near an upper-extremity location of the user, and
provide an estimate of blood pressure or cardiac output to account for at least one of the following: autonomic response changes in the circulatory system, cardiac output distribution to the head region versus another body portion of the user, variations in total peripheral resistance (TPR) in at least one of the upper-extremity and lower-extremity locations that influence local pressure, and a distance-based parameter indicative of differing lengths in an artery segment extending towards the upper-extremity location and an artery segment extending towards the lower-extremity location.

11. A method comprising:
using a sensor to obtain, at or near a lower-body (or lower-extremity) location of the user, time-related data indicative of speed or transit time of a propagating pressure wave while the wave travels in an artery and down a leg of the user; and
correlating information, by use of a circuit, corresponding to or derived from the time-related data in a time synchronous manner with upper-body or lower body cardiovascular information, the upper-body or lower-body cardiovascular information corresponding to or derived from hemodynamic output from the user by another sensor located at or near an upper-extremity location or lower-extremity of the user.
